(19) United States
(12) Patent Application Publication
(10) Pub. No.: US 2012/0157938 A1
(43) Pub. Date: Jun. 21, 2012

(54) PUNCTAL PLUG WITH DRUG CORE RETENTION FEATURES

(76) Inventors: Jason M. Tokarski, Jacksonville, FL (US); Victor Lust, Jacksonville, FL (US); Chris Lyons, Jacksonville, FL (US); Jeffrey Miller, St. Augustine, FL (US); Phillip Parnell, SR., Jacksonville, FL (US); Günter Solms, Jacksonville, FL (US)

(21) Appl. No.: 13/246,420

(22) Filed: Sep. 27, 2011

Related U.S. Application Data
(60) Provisional application No. 61/423,841, filed on Dec. 16, 2010.

(51) Int. Cl. A61F 9/00

(52) U.S. Cl. 604/294

(57) ABSTRACT

Disclosed are lacrimal inserts and their method of use for delivery of of medication to the eye. The plug includes a body portion sized to pass through a lacrimal punctum and be positioned within a lacrimal canaliculus of the eyelid. The plug may contain a core, or reservoir, at least partially within the body portion comprising a therapeutic agent that is configured to controlled release into the eye and is configured to release medication via a designated port, valve, or orifice in the insert housing and inhibits diffusion of medication via the housing itself The core may include retention features to ensure that it remains securely located within the lacrimal insert over its usable life.

This patent application claims the benefit of U.S. Provisional Application Ser. No. 61/423,841, filed Dec. 12, 2010.

### BACKGROUND OF THE INVENTION

1. Field of the Invention

The present invention relates to an ophthalmic insert and method for the release of medication to the eye for the treatment of eye disorders. More specifically, the present invention relates to punctal plugs sized to pass through a lacrimal punctum and be positioned within a lacrimal canaliculus of the eyelid and which contains medication for controlled release into the eye in which the punctal plug is placed. The punctual plug includes features to ensure containment of a drug core therein.

2. Discussion of the Related Art

Active agents frequently are administered to the eye for the treatment of ocular diseases and disorders. Conventional means for delivering active agents to the eye involve topical application to the surface of the eye. The eye is uniquely suited to topical administration because, when properly constituted, topically applied active agents can penetrate through the cornea and rise to therapeutic concentration levels inside the eye. Active agents for ocular diseases and disorders may be administered orally or by injection, but such administration routes are disadvantageous in that, in oral administration, the active agent may reach the eye in too low a concentration to have the desired pharmacological effect and their use is complicated by significant, systemic side effects and injections pose the risk of infection.

The majority of ocular active agents are currently delivered topically using eye drops which, though effective for some applications, are inefficient. When a drop of liquid is added to the eye, it overfills the conjunctival sac, the pocket between the eye and the lids, causing a substantial portion of the drop to be lost due to overflow of the lid margin onto the cheek. In addition, a substantial portion of the drop that remains on the ocular surface is drained into the lacrimal puncta, diluting the concentration of the drug.

To compound the problems described above, patients often do not use their eye drops as prescribed. Often, this poor compliance is due to an initial stinging or burning sensation caused by the eye drop. Certainly, instilling eye drops in one's own eye can be difficult, in part because of the normal reflex to protect the eye. Therefore, sometimes one or more drops miss the eye. Older patients may have additional problems instilling drops due to arthritis, unsteadiness, and decreased vision. Pediatric and psychiatric patient populations pose difficulties as well.

It is known to use devices that may be inserted into one or more of an orifice of an individual's eye, such as a lacrimal punctum, to deliver active agents. One disadvantage of using such devices to deliver agents is that much of the agent may delivered in an initial, large bolus upon insertion of the device into the eye rather than a more linear delivery of the agent over time.

Prior topical sustained release systems include gradual release formulations, either in solution or ointment form, which are applied to the eye in the same manner as eye drops but less frequently. Such formulations are disclosed, for example, in U.S. Pat. No. 3,826,258 issued to Abraham and U.S. Pat. No. 4,923,699 issued to Kaufman. Due to their method of application, however, these formulations result in many of the same problems detailed above for conventional eye drops. In the case of ointment preparations, additional problems are encountered such as a blurring effect on vision and the discomfort of the sticky sensation caused by the thick ointment base.

Alternately sustained release systems have been configured to be placed into the conjunctival cul-de-sac, between the lower lid and the eye. Such units typically contain a core drug-containing reservoir surrounded by a hydrophobic copolymer membrane which controls the diffusion of the drug. Examples of such devices are disclosed in U.S. Pat. No. 3,618,604 issued to Ness, U.S. Pat. No. 3,626,940 issued to Zaffaroni, U.S. Pat. No. 3,845,770 issued to Theeuwes et al., U.S. Pat. No. 3,962,414 issued to Michaels, U.S. Pat. No. 3,993,071 issued to Higuchi et al., and U.S. Pat. No. 4,014,335 issued to Arnold. However, due to their positioning, the units may be uncomfortable and poor patient acceptance is again encountered.

### BRIEF DESCRIPTION OF THE DRAWINGS

The foregoing and other features and advantages of the invention will be apparent from the following, more particular description of preferred embodiments of the invention, as illustrated in the accompanying drawings.

FIG. 1 is a plan view of an illustrative example of a lacrimal insert in accordance with the present invention.

FIG. 2 is a partial cross-sectional view of an illustrative example of a lacrimal insert, showing a drug core having locking rings to anchor the drug core in a cavity within the insert housing in accordance with the present invention.

FIG. 3 is a cross-sectional view of an illustrative example of a lacrimal insert, that has a cavity within the housing of the insert that is tapered from its narrowest at the opening and widest at the base of the reservoir in accordance with the present invention.

FIG. 4 is a cross-sectional view of another configuration for a lacrimal insert wherein the cavity in the housing has a portion with straight walls and a portion with tapered walls in accordance with the present invention.

FIG. 5 illustrates a partial cross-sectional view of another configuration for a lacrimal insert wherein the drug core includes a protruding, spiral feature to aid in retention of the core within the drug core cavity in the housing in accordance with the present invention.

FIG. 6 is a partial cross-sectional view of an illustrative example of a lacrimal insert, showing the drug core having multiple locking rings to anchor the drug core in a cavity within the insert housing in accordance with the present invention.

FIG. 7 illustrates an exemplary lacrimal insert with a pattern of inwardly and outwardly tapering inner surfaces in the cavity for retaining the drug core therein in accordance with the present invention.

FIG. 8 is a partial cross-sectional view of an illustrative example of a lacrimal insert, showing the drug core having multiple locking rings to anchor the drug core in a cavity within the insert housing in accordance with the present invention.

FIG. 9 illustrates an exemplary lacrimal insert with a cavity within the housing having a semi-spherical portion at its base in accordance with the present invention.

FIG. 10 illustrates a partial cross-sectional view of another configuration for a lacrimal insert wherein the drug core includes multiple protruding, spiral features to aid in retention of the core within the drug core cavity in the housing in accordance with the present invention.

FIG. 11 illustrates an exemplary lacrimal insert having a drug core with multiple spiral features and an anchor head securing the core within the drug core cavity in the housing in accordance with the present invention.

FIG. 12 illustrates an exemplary lacrimal insert having a drug core with multiple locking spiral features and an anchor head securing the core within the drug core cavity in the housing in accordance with the present invention.

FIG. 13 illustrates another exemplary embodiment of a lacrimal insert according to the present invention wherein a locking rings is provided to aid in retention of the drug core within the housing.

FIG. 14 illustrates another exemplary embodiment of a lacrimal insert according to the present invention wherein locking rings are provided to aid in retention of the drug core within the housing.

### DETAILED DESCRIPTION OF THE PREFERRED EMBODIMENTS

Punctal plugs have been in use for decades now to treat conditions of dry eye. More recently they have gained attention for use as drug delivery systems for the treatment of ocular diseases and conditions. Several challenges exist with formulating a drug to release at the desired daily rate and/or dose that will give efficacy while limiting adverse events.

Diffusion based drug delivery systems are characterized by the drug release rate which is dependent on its diffusion through an inert water insoluble membrane barrier. There are basically two diffusion designs: reservoir devices and matrix devices. Reservoir devices are those in which a core of drug is surrounded by polymeric membrane. The nature of the membrane determines the rate of release of drug from the system. The process of diffusion is generally described by a series of equations governed by Fick's first law of diffusion. A matrix device typically consists of a drug dispersed homogenously throughout a polymer.

Reservoir and matrix drug delivery systems are considered diffusion based sustained release systems and constitute any dosage form that provides medication over an extended period of time. The goal of a sustained release system is to maintain therapeutic levels of a drug for an extended period and this is usually accomplished by attempting to obtain zero-order release from the sustained release system. Sustained release systems generally do not attain this type of release profile but try to approximate it by releasing in a slow first order manner. Over time, the drug release rate from reservoir and matrix sustained release systems will decay and become non therapeutic.

Zero-order drug release constitutes drug release from a drug delivery system at a steady sustained drug release rate, that is, the amount of drug that is released from the drug delivery system over equal time intervals does not decay and remains at the therapeutic level. This “steady sustained release drug delivery system” is referred to as a zero-order drug delivery system and has the potential to provide actual therapeutic control by its controlled release.

Another drug release profile is referred to as pulsatile drug delivery. Pulsatile drug delivery is intended to release a therapeutic amount of a therapeutic agent at regular intervals.

Without regard to the desired drug release profile of the lacrimal insert device, the different therapeutic agents that are desirable for use in a lacrimal insert may react or behave differently from one another. Some therapeutic agents may be soluble in, effuse through, or react with various materials that may be used to construct the punctal plug (a term that may be used interchangeably throughout this specification with the term lacrimal insert).

It has been found that with certain therapeutic agents, it may be desirable to create a barrier layer between an active agent containing material contained in a reservoir within the lacrimal insert and the interior surface of the housing that defines the reservoir. Moreover, it has been found that retention of the drug core may be aided by the selection of the geometric configuration of the lacrimal insert or with the addition of various anchoring features, as well as retention of the insert in the lacrimal puncta. Alternately, these features may be used alone or in combination, or varied. For example, the barrier layer may be disposed on the external surface of the punctal plug to inhibit diffusion of the therapeutic agent via the housing of the punctal plug and to inhibit the infusion of lacrimal fluid into the reservoir containing the active agent containing material.

The drug core that is inserted into a lacrimal insert for purposes of drug delivery may be required to remain in situ for an extended period of time (ranging from hours to perhaps as long as months). During that time, the constant exposure of the active pharmaceutical insert (API) that comprises the drug core to lacrimal fluid may cause the core to become dislodged or inappropriately placed within the insert. In some cases, the drug core may possibly be ejected from the lacrimal insert due to the pressure placed on the housing by the lacrimal puncta within which the insert is placed when in-use.

In exemplary embodiments where the drug core is generally a solid or semi solid formulation of material, it may grow smaller in size as it elutes into the lacrimal fluid to provide treatment. As the drug core (or API) becomes smaller, the chances for the insert to escape from the lacrimal insert increases. Not only could such a situation cause discomfort to the patient, but the exposure to the entire API to lacrimal fluid may result in inaccurate dosing of the active ingredient. Further, where the API remains in the drug core cavity within the lacrimal insert, care must be taken to ensure that the amount of surface area of the API that is exposed to lacrimal fluid remain relatively constant until the API has been depleted.

In current attempts to address these concerns with existing lacrimal inserts, it has been found that various mechanical means for retaining the drug core within the drug core cavity of a lacrimal insert may provide substantially superior performance of the device over its usable lifetime. Moreover, a device in which the drug core has been fitted with features to aid in its retention within the lacrimal insert may be more likely to provide accurate dosing to the patient with reduced risk of unintended ejection of the API from the housing.

Typically, the drug core comprises a formulation of the API and excipients. Although not limited to such, a preferred API may comprise latanoprost, bimatoprost, travoprost, cyclosporine, bromfenac, and/or levofloxacin and use excipients including ethylene vinyl acetate (EVA) and polycaprolactone (PCL). Combined together, the resulting drug core may be placed in a cavity within a punctal plug (lacrimal insert). The active therapeutic ingredient then elutes out of an exposed or open end of the lacrimal insert over time according to any one or more of the possible drug delivery profiles (zero order, first order, etc. and combinations thereof).

To illustrate these concepts with greater specificity, we now turn to the figures. These figures are meant to be instructive, but not exhaustive of the possible structure and materials of the exemplary embodiments of the present invention and wherein similar reference numerals refer to similar structure.

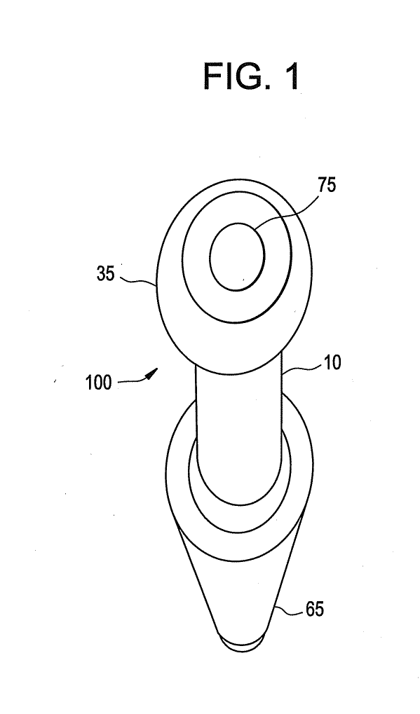

An exemplary punctal plug configured for release of a therapeutic agent is illustrated in FIG. 1. Shown is punctal plug 100 having a rounded “arrowhead” like first end 65 designed to permit insertion of the device into a lacrimal punctum of a patient. The punctal plug 100 has a housing 10 and a second end having a flange (or lip) 35 that may engage the surface of the eye and inhibit the punctal plug 100 from being completely inserted into the lacrimal punctum. The drug is intended to elute from opening 75.

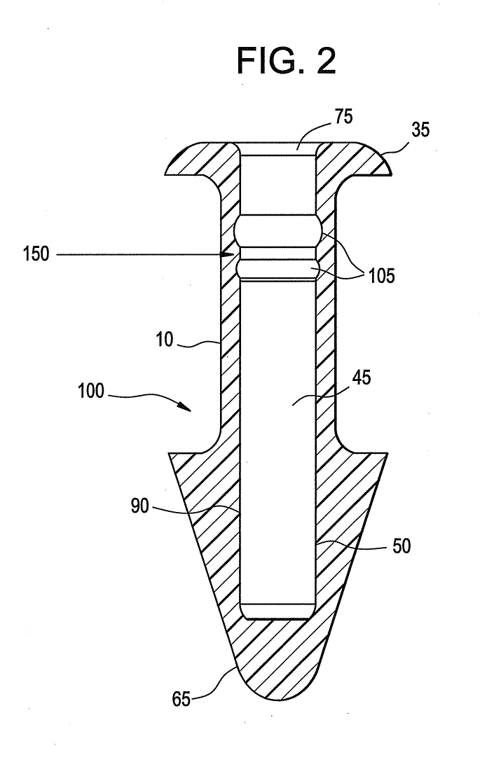

FIG. 2 illustrates an exemplary embodiment of the punctal plug 100 shown in FIG. 1 in which a drug core 90 is inserted into a cavity 45 in the housing 10. To retain the drug core 90 within the cavity 45, retention features 150 are disposed between the drug core 90 and the inner surface 50 of the housing 10 that defines the drug core cavity or reservoir. The punctal plug 100 has an interior cavity, or reservoir 45, configured to contain a quantity of therapeutic agent or agent that is contained in a carrier or other material, generally referred to herein as an active-agent containing material or active pharmaceutical insert (API) when it is in the form of a “drug core” that may be inserted into a punctal plug or formed within the drug core cavity of the plug in situ. The reservoir 45 may, therefore, be defined by the interior surface of the housing 50. The retention features 150, in this exemplary embodiment, comprise one or more locking rings 105 disposed between the drug core 90 and the inner surface 50 of the housing that defines the cavity 45. Alternately, and unless otherwise noted, the drug core could be formed in situ.

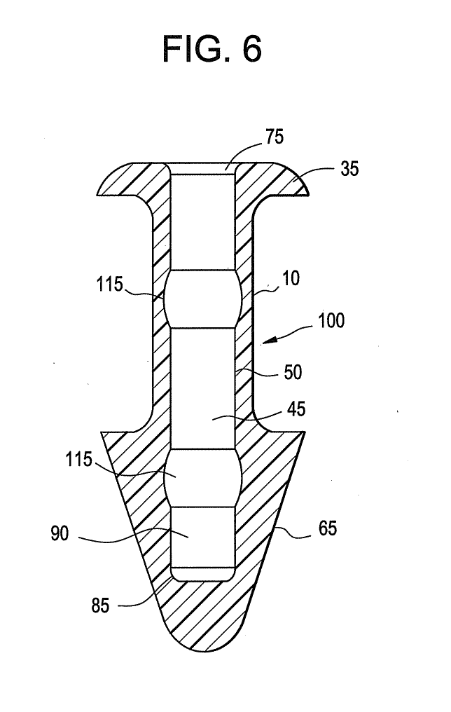

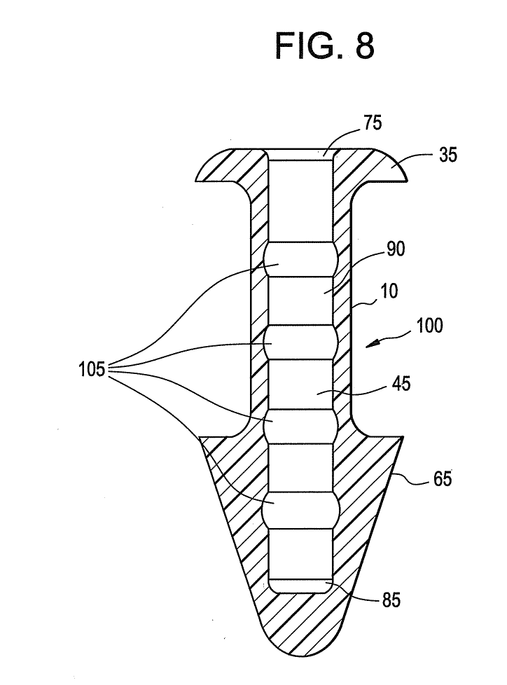

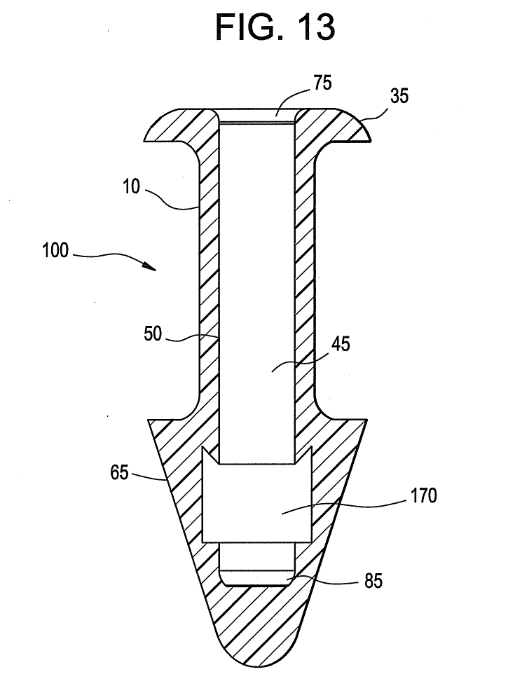

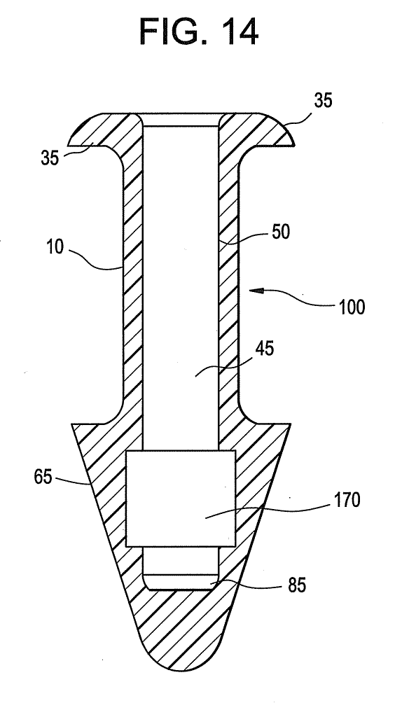

In other exemplary embodiments illustrated in FIGS. 6 and 8, multiple locking rings 115, 105 may be used to hold the drug core 90 in the cavity 45 by frictional forces. As illustrated in FIGS. 13 and 14, the drug core itself may include large locking rings 170, or locking rings in a variety of configurations to increase the frictional forces that retain the drug core in the cavity.

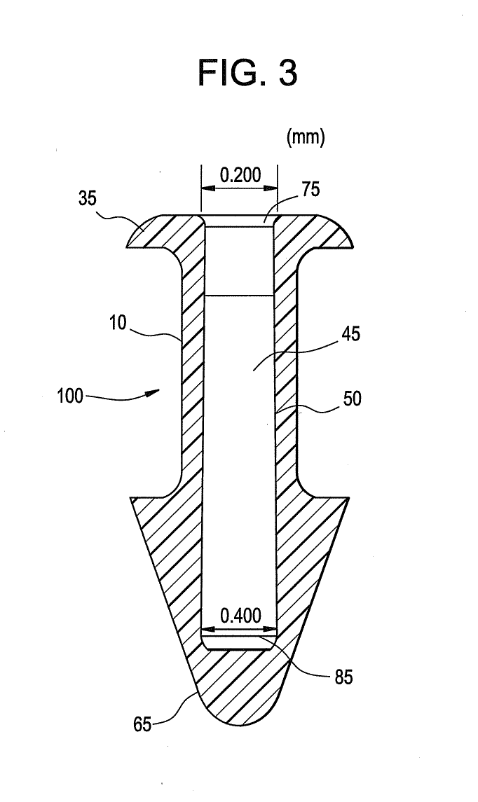

FIG. 3 illustrates punctal plug 100 according to another exemplary embodiment of the present invention. In this exemplary embodiment, the inner surface of the housing 50 that defines the drug core cavity 45 is tapered from the opening 75 where the drug is intended elute from to the base of the cavity 85. By narrowing the cavity 45 from the opening 75 to the base 85, the drug core therein forms a natural wedge. This geometry effectively captures the drug core within the housing 50 and minimizes the possibility of displacement over time. By way of a non-limiting example, in a typically sized lacrimal insert of 2.5 mm in length, the tapered chamber may have an opening of about 0.20 mm in diameter at the open end of the device 75 and of about 0.40 mm at the base 85 of the cavity 45.

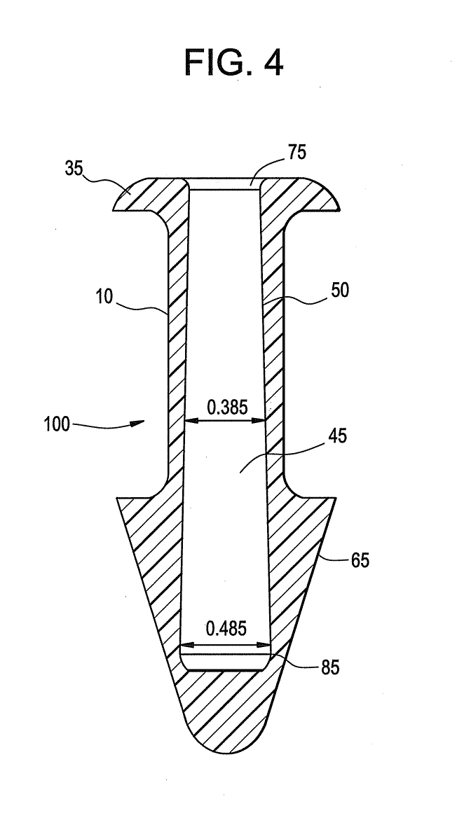

FIG. 4 illustrates another exemplary embodiment of the present invention in which a portion of the inner surface of the housing 50 that defines the drug core cavity 45 is tapered from about its midpoint between the opening 75 of the cavity 45 and the base 85 of the cavity 45. This geometry effectively captures a portion of the drug core within the housing and minimizes the possibility of displacement over time. Since the drug core is generally solid, capturing a portion of it is generally sufficient to ensure that it remains properly located over the usable life of the lacrimal insert. By way of another non-limiting example, in a typically sized lacrimal insert of 2.5 mm in length, the tapered chamber may have an opening of about 0.385 mm in diameter at the midpoint of the cavity 45 and of about 0.485 mm at the base 85 of the cavity 45.

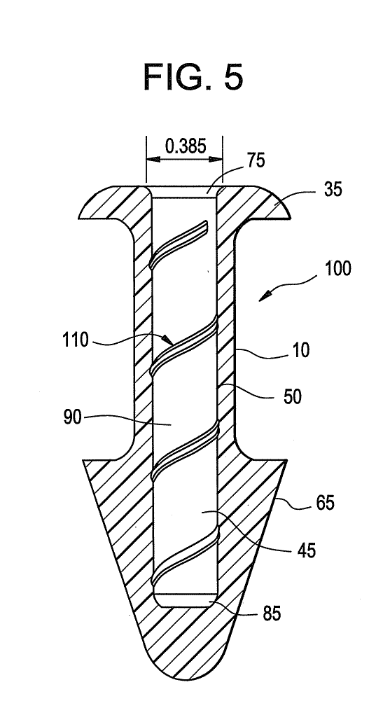

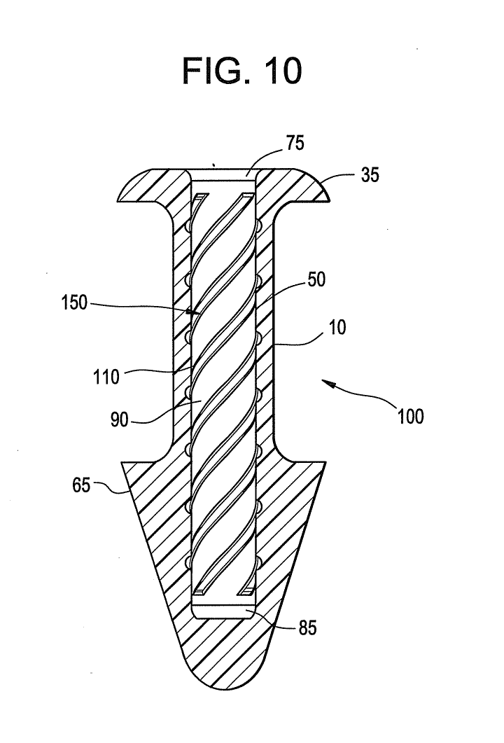

FIG. 5 illustrates an exemplary embodiment of the punctal plug 100 shown in FIG. 1 in which the drug core is inserted into a cavity 45 in the housing 10. To retain the drug core 90 within the cavity 45, a retention feature is disposed between the drug core and the inner surface 50 of the housing 10 that defines the drug core cavity or reservoir. In this exemplary embodiment, the retention feature may comprise a spiral element 110. By use of the spiral element 110, the drug core 90 is firmly held in place by frictional forces of the spiral element 110 against the inner surface of the housing 50. Alternately, and unless otherwise noted, the drug core could be formed in situ. Further, multiple spiral features may be used, as shown in FIG. 10.

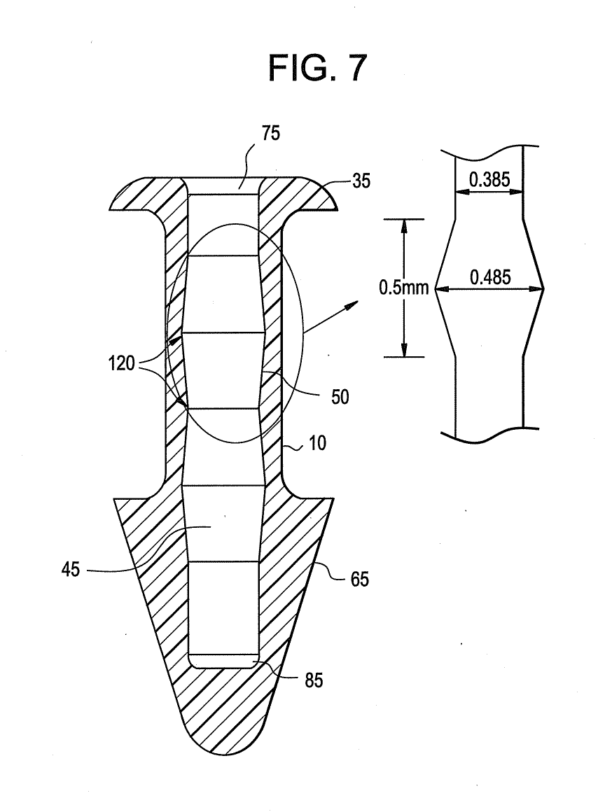

In FIG. 7, the drug core cavity 45 is shown having a plurality of inwardly and outwardly tapering surfaces. Such a configuration may prevent displacement of the drug core as it reduces in size during its usable lifetime.

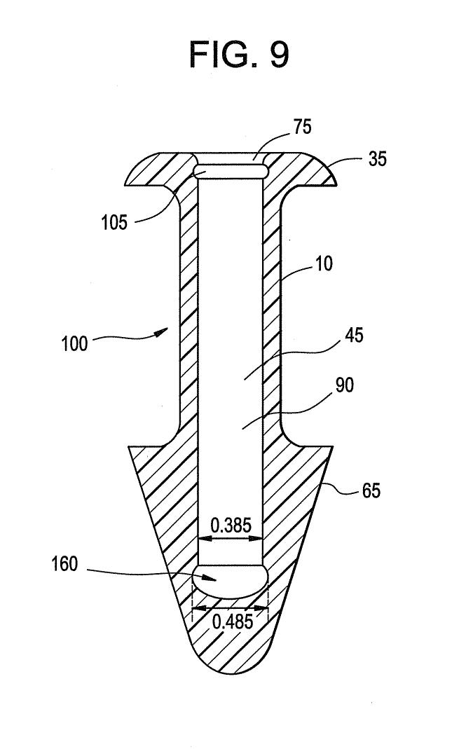

FIG. 9 illustrates another exemplary embodiment of the present invention where the drug core 90 may be formed with an anchor head 160 that may take the form of a bulbous retention feature. Although shown as semispherical for purposes of illustration, many shapes may be used for this feature, as it may take advantage of the additional space provided within the arrowhead portion 65 of the lacrimal insert 100. Further, the reservoir or cavity 45 may be formed with an anchor head to permit the insertion of a pre-formed drug core 90 therein or to permit the in situ manufacturing of the core therein.

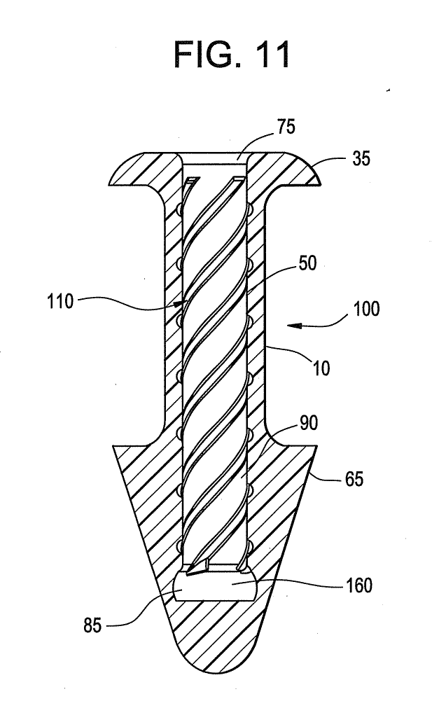

Various combinations of retention features may be used to provide greater certainly that the drug core will remain properly placed during use. As illustrated in FIG. 11, the anchor head retention feature 160 described and shown in FIG. 9 may be combined with other features, such as the multiple spiral elements 110 shown in FIG. 10.

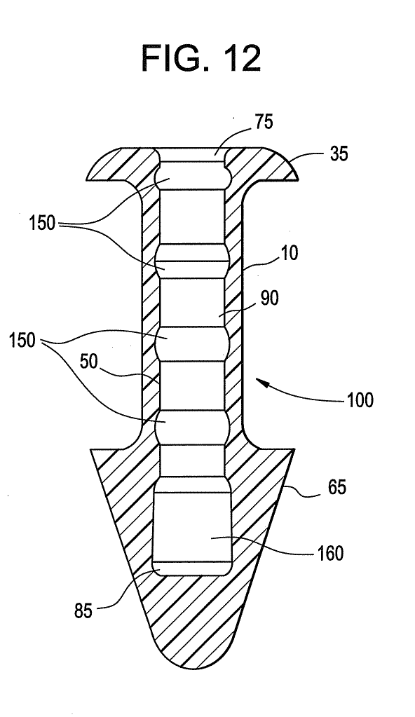

In another example of a combination of features, FIG. 12 illustrates the use of multiple locking rings 150 (described with respect to FIGS. 2, 6, and 8) in combination with an anchor head 160 having a non-spherical profile.

A punctal plug configuration according to the exemplary embodiments of the present invention may provide for containment of a solid, semi-solid, suspension, particulate and some gel formulations. Exemplary materials for construction of the punctal plug, or other components of the plug that are intended for containing the therapeutic agent or active-agent containing material, or are intended to inhibit the infusion of lacrimal fluid into the reservoir of the lacrimal insert by means other than orifices, ports, membranes, or valves included for the purpose of regulating the delivery of therapeutic agent or active ingredient may comprise Polytetrafluoroethylene, Fluorinatedethylenepropylene, Perfluoroalkoxy, Polyvinylidene Fluoride, Tetrafluoroethylene, Hexafluoropropylene and Vinylidene Fluoride, Ethylenetetrafluoroethylene, Chloro Trifluoro Ethylene/Ethylene Copolymer, Polyethylene Terepthalate Polyester, Polyetheretherketone, Nylon 6/6, Nylon 11, Nylon 12, Pebax, Polyethylene, Ultra high molecular weight polyethylene, Ultra low molecular weight polyethylene, High molecular weight polyethylene, High density polyethylene, High density cross-linked polyethylene, Cross-linked polyethylene, Medium density polyethylene, Low density polyethylene, Linear low density polyethylene, Very low density polyethylene, Polypropylene, Polycaprolactone, Cellulose and cellulose derivatives, Cellulose Acetate, Polycarbonate, Cyanoacrylate, Eudragit, Polyimide, Polyamide, Ethylene Vinyl Acetate, Polyurethane, Polysulfone, Polyetherimide, Polytheresulfone, Styrene butadiene Rubber, Polyphenylene Sulfide, Polyphenylene Oxide, Ethylene Propylene Diene Monomer (Rubber), Zeonor, Zeonex, Parylene, Parylene N, Parylene C, Parylene D, Parylene AF-4, Parylene SF, Parylene HT, Parylene A, Parylene AM, Parylene VT-4, Parylene CF, and Parylene X, Polyvinyl alcohol, Polyvinyl acetate, Polyvinyl chloride, Polyisobutylene, Fluro silicones, Liquid curable perfluoropolyether, and Polystyrene.

MDPE (medium density polyethylene) is defined by a density range of 0.926-0.940 g/cm³. MDPE can be produced by chromium/silica catalysts, Ziegler-Natta catalysts or metallocene catalysts. MDPE has good shock and drop resistance properties. It also is less notch sensitive than HDPE, stress cracking resistance is better than HDPE. MDPE is typically used in gas pipes and fittings, sacks, shrink film, packaging film, carrier bags and screw closures.

LLDPE (linear low density polyethylene) is defined by a density range of 0.915-0.925 g/cm³. LLDPE is a substantially linear polymer with significant number of short branches, commonly made by copolymerization of ethylene with short-chain alpha olefins (for example, 1-butene, 1-hexene and 1-octene). LLDPE has higher tensile strength than LDPE, it exhibits higher impact and puncture resistance than LDPE. Lower thickness (gauge) films can be blown, compared with LDPE, with better environmental stress cracking resistance but it not as easy to process. LLDPE is used in packaging, particularly film for bags and sheets. Lower thickness may be used compared to LDPE. Cable covering, toys, lids, buckets, containers and pipe. While other applications are available, LLDPE is used predominantly in film applications due to its toughness, flexibility and relative transparency.

LDPE (low density polyethylene) is defined by a density range of 0.910-0.940 g/cm³. LDPE has a high degree of short and long chain branching, which means that the chains do not pack into the crystal structure as well. It has, therefore, less strong intermolecular forces as the instantaneous-dipole induced-dipole attraction is less. This results in a lower tensile strength and increased ductility. LDPE is created by free radical polymerization. The high degree of branching with long chains gives molten LDPE unique and desirable flow properties. LDPE is used for both rigid containers and plastic film application such as plastic bags and films wraps.

VLDPE (very low density polyethylene) is defined by a density range of 0.880-0.915 g/cm³. VLDPE is a substantially linear polymer with high levels of short-chain branches, commonly made by copolymerization of ethylene with short-chain alpha-olefins (for example, 1-butene, 1-hexene and 1-octene). VLDPE is most commonly produced using metallocene catalysts due to the greater co-monomer incorporation exhibited by these catalysts. VLDPEs are used for hose and tubing, ice and frozen food bags, food packaging and stretch wrap as well as impact modifiers when blended with other polymers.

Recently much research activity has focused on the nature and distribution of long chain branches in polyethylene. In HDPE a relatively small number of these branches, perhaps 1 in 100 or 1,000 branches per backbone carbon, may significantly affect the rheological properties of the polymer.

As used herein, the term “active agent” refers to an agent capable of treating, inhibiting, or preventing a disorder or a disease. Exemplary active agents include, without limitation, pharmaceuticals and nutraceuticals. Preferred active agents are capable of treating, inhibiting, or preventing a disorder or a disease of one or more of the eye, nose and throat.

As used herein, the term “punctal plug” refers to a device of a size and shape suitable for insertion into the inferior or superior lacrimal canaliculus of the eye through, respectively, the inferior or superior lacrimal punctum. Exemplary and illustrative devices are disclosed in U.S. Pat. No. 6,196,993 and U.S. Published Patent Application No. 20090306608A1, both of which are hereby incorporated by reference in their entireties.

As used herein, the term “opening” refers to an opening in the body of a device of the present invention of a size and shape through which the active agent can pass. Preferably, only the active agent can pass through the opening. The opening may be covered with a membrane, mesh, grid or it may be uncovered. The membrane, mesh, or grid may be one or more of porous, semi-porous, permeable, semi-permeable, and biodegradable.

The devices of the present invention have a reservoir in which is found an active agent-containing material and an active agent therein. The active agent may be dispersed throughout the active agent-containing material or dissolved within the material. Alternately, the active agent may be contained in inclusions, particulates, droplets, or micro-encapsulated within the material. Still as another alternate, the active agent may be covalently bonded to the material and released by hydrolysis, enzymatic degradation and the like. Yet as another alternate, the active agent may be in a reservoir within the material.

In accordance with one exemplary embodiment of the present invention, the active agent may be released in a controlled manner, meaning over a period of time by using an active agent-containing material in which the agent is present in a continuous concentration gradient throughout the material or by using a discontinuous concentration gradient. This is in contrast to a device that exhibits a “burst” or immediate release upon insertion of an amount of active agent that is greater than the average release rate over time. The structure recited herein, however, may be applied with equal success in devices designed to release the therapeutic agent or active-agent containing material according to either profile.

Without being bound to any particular theory, it is believed that an active agent-containing material that does not undergo significant chemical degradation during the time desired for the release of active agent will release the agent by diffusion through the matrix to a device's release surfaces, meaning surfaces of the active agent-containing material in contact with a person's body fluid. According to Fick's Law, the diffusive transport or flux, J, of the agent through the active agent-containing material is governed at each point and each time by the local concentration gradient, the diffusivity of the active agent with the material D, and the spatial variation of the cross-sectional geometry of the device.

The local gradient may be controlled by placing more active agent at one location in the active agent-containing material relative to another location. For example, the concentration profile may be a continuous gradient from one end of the material to the other. Alternately, the matrix may be have a discontinuous gradient, meaning that one section of the material has a first concentration and the concentration abruptly changes to a second, different concentration in an adjacent section of the matrix, such as that illustrated in the alternate exemplary embodiments illustrated in FIGS. 1 and 4 as being contained in the drug impermeable housing. The diffusivity for the active agent may also be spatially controlled by varying one or more of the chemical composition, porosity, and crystallinity of the active agent-containing material.

Additionally, the spatial variation of the material's cross-sectional geometry may be used to control diffusivity. For example, if the material was in the form of a straight rod that has a uniform active agent concentration, diffusivity will be reduced when the area at the open end of the material is significantly smaller than the average of the entire material. Preferably, the material area at the open end of the device is no more than about one-half of the average cross sectional area of the material, meaning the cross section determined perpendicular to the primary dimension of active agent transport use.

One of ordinary skill in the art will recognize that, depending on how one varies one or more of the local concentration gradient, the diffusivity of the active agent from the material, and the spatial variation of the cross-sectional geometry of the device, a variety of release profiles may be obtained including, without limitation first order, second order, biphasic, pulsatile and the like. For example, either or both of the active agent concentration and diffusivity may increase from the surface to the center of the active agent-containing material in order to achieve more initial release. Alternately either or both may be increased or decreased and then increased again within the material to achieve a pulsatile release profile. The ability to achieve a variety of release profiles by varying the local concentration gradient, the diffusivity of the active agent, and the spatial variation of the cross-sectional geometry may eliminate the need for rate-limiting membranes in the device.

The exemplary devices of the present invention comprise a reservoir within the body, and the reservoir includes at least one active agent-containing material, as shown in the exemplary embodiments of FIGS. 1 and 2. The body 10 is preferably impermeable to the active agent, meaning only an insubstantial amount of active agent can pass there through, and the body has at least one opening 75 through which the active agent is released. An active agent-containing material useful in the exemplary devices of the present invention is any material that is capable of containing the active agent, does not alter the chemical characteristics of the active agent, and does not significantly chemically degrade or physically dissolve when placed in contact with ocular fluids. Preferably, the active agent-containing material is non-biodegradable, meaning that it does not degrade to a substantial degree upon exposure to biologically active substances typically present in mammals. Additionally, the active agent-containing material is capable of releasing the active agent by one or more of diffusion, degradation, or hydrolyzation. Preferably, the active agent-containing material is a polymeric material, meaning that it is a material made of one or more types of polymers.

When the active agent-containing material is combined with the active agent, thereby forming the material included in the reservoir or cavity 45, the material may also comprise one or more materials that are insoluble in water and non-biodegradable, but from which the active agent may diffuse. For example, if the active agent-containing material is a polymeric material, the material may be composed of one or more polymers that are insoluble in water and non-biodegradable.

Suitable polymeric materials for the active agent-containing material include hydrophobic and hydrophilic absorbable and non-absorbable polymers. Suitable hydrophobic, non-absorbable polymers include ethylene vinyl alcohol (“EVA”), fluorinated polymers include polytetrafluoroethylene (“PTFE”) and polyvinylidene fluoride (“PVDF”), polypropylene, polyethylene, polyisobutylene, nylon, polyurethanes, polyacrylates and methacrylates, polyvinyl palmitate, polyvinyl stearates, polyvinyl myristate, cyanoacrylates, epoxies, silicones, copolymers thereof with hydrophobic or hydrophilic monomers, and blends thereof with hydrophilic or hydrophobic polymers and excipients.

Hydrophilic, non-absorbable polymers useful in the present invention include cross-linked poly(ethylene glycol), poly(ethylene oxide), polypropylene glycol), poly(vinyl alcohol), poly(hydroxyethyl acrylate or methacrylate), poly(vinylpyrrolidone), polyacrylic acid, poly(ethyloxazoline), and poly(dimethyl acrylamide), copolymers thereof with hydrophobic or hydrophilic monomers, and blends thereof with hydrophilic or hydrophobic polymers and excipients.

Hydrophobic, absorbable polymers that may be used include aliphatic polyesters, polyesters derived from fatty acids, poly(amino acids), poly(ether-esters), poly(ester amides), polyalkylene oxalates, polyamides, poly(iminocarbonates), polycarbonates, polyorthoesteres, polyoxaesters, polyamidoesters, polyoxaesters containing amine groups, phosphoesters, poly)anhydrides), polypropylene fumarates, polyphosphazenes, and blends thereof. Examples of useful hydrophilic, absorbable polymers include polysaccharides and carbohydrates including crosslinked alginate, hyaluronic acid, dextran, pectin, hydroxyethyl cellulose, hydroxy propyl cellulose, gellan gum, guar gum, keratin sulfate, chondroitin sulfate, dermatan sulfate, proteins including collagen, gelatin, fibrin, albumin and ovalbumin, and phospholipids including phosphoryl choline derivatives and polysulfobetains.

More preferably, the active agent-containing material comprises a polymeric material that is polycaprolactone. Still more preferably, the material comprises poly(epsilon-caprolactone), and ethylene vinyl acetate of molecular weights between about 10,000 and 80,0000. About 0 to about 100 weight percent polycaprolactone and about 100 to about 0 weight percent of the ethylene vinyl acetate are used based on the total weight of the polymeric material and, preferably, about fifty (50) percent each of polycaprolactone and ethylene vinyl acetate is used.

The polymeric material used is preferably greater than about ninety-nine (99) percent pure and the active agents are preferably greater than about ninety-seven (97) percent pure. One of ordinary skill in the art will recognize that in compounding, the conditions under which compounding is carried out will need to take into account the characteristics of the active agent to ensure that the active agents do not become degraded by the process. The polycaprolactone and ethylene vinyl acetate preferably are combined with the desired active agent or agents, micro-compounded, and then extruded.

The release kinetics of the therapeutic agent or active-agent containing material may be controlled via spatial gradients of the properties of degradability and drug permeability of the active agent-containing material. For example, in those cases in which drug release kinetics are dominated by the rate of material degradation, a spatial degradation in the material chemistry including polylactide-glycolide copolymers of differing monomer ratios, adjacent polyglycolide and polycaprolactone layers and the like, results in spatial gradients and varied release rates as the material degradation front moves through the device. By way of further example, a material may erode more slowly initially in a first, outer material and more quickly in a second, inner material to achieve phased release kinetics.

In the case of a non-degradable material that elutes the active agent solely through diffusion-dominated mechanisms, spatial gradients in the material's permeability may control release kinetics beyond what is possible with a homogeneous material. In the diffusion-dominated mechanism, the material permeability controls release kinetics and is influenced by the material's porosity as well as the active agent solubility and diffusivity. By forming an active agent-loaded layer of an outer material with a higher permeability, the active agent elution may be controlled to be more linear with less burst effect than that which is otherwise achieved with a single, homogeneous, diffusion material.

The spatial gradients in biodegradability or permeability may be combined with continuous or step-wise gradients in the active agent loading profile. For example, a punctal plug material core having an outer segment loaded with a low active agent concentration and with a relatively low active agent permeability may be adjacent to an inner material segment loaded with a high agent concentration and with a relatively high active agent permeability, which combination achieves release kinetics unobtainable with a homogeneous material ad homogeneous active agent loading. The initial burst release is reduced and the release of the last active agent content is accelerated relative to a conventional homogeneous active agent loaded device.

Phase-separated inclusions may be used to control one or both of diffusive and degradative kinetics of the active agent-containing material. For example, water soluble polymers, water soluble salts, materials with a high diffusivity for the active agent and the like may be used as destabilizing inclusion to enhance degradation or diffusion rates. When the hydrolysis front reaches an inclusion, the inclusion rapidly dissolves and increases porosity of the active agent-containing material. The inclusions may be incorporated as gradients or layers that allow additional tailoring of the release profile.

As another alternate, a network of destabilizing inclusions may be used. When used in a non-biodegradable active agent-containing material, these inclusions form islands within the material that may possess high diffusivity for the active agent. Useful inclusions will have a higher diffusivity for the active agent than the active agent-containing material. Examples of such inclusions include propylene glycol, silicone oil, immiscible dispersed solids such as a polymer or wax and the like. As yet another example, an inclusion that acts to adsorb water, swell the active agent-containing material and increase local diffusion kinetics may be used.

As still another alternate, stabilizing inclusions that have a low active agent diffusivity are used. These inclusions act to form a barrier that slows diffusive transport of the active agent in the vicinity of the inclusion. The overall effect is a reduction of active agent permeability in a base material that is otherwise the same. Example of such inclusions include micro to nano-sized silicate particles dispersed through the base material of one or both of polycaprolactone and ethylenecovinylacetate homogeneously or in continuous step-wise gradients.

The present invention encompasses numerous devices for the delivery of active agents to the eye, each having various features and advantages. For example, certain devices may have a body with a first end, a second end, and a lateral surface extending between the two ends. The lateral surface preferably has an outer diameter that is substantially circular in shape and, thus, the body preferably has a cylindrical shape. A portion of the lateral surface of certain of the devices preferably has an outer diameter that is greater than the outer diameter of the remainder of the lateral surface as illustrated in FIGS. 5 and 6. The enlarged portion may be any size or shape, and may be present on any part of the lateral surface, in punctal plug embodiments, the enlarged portion is of a size so that it at least partially anchors the punctal plug in the lacrimal canaliculus and preferably, the enlarged portion is at one end of the plug. Further, it has been found that by selecting a cross-sectional profile geometry of this nature may improve the retention of the drug-containing core present in the reservoir. One ordinarily skilled in the art will recognize that any of a wide variety of shapes may be possible.

The body of the punctal plugs of the present invention may take any shape and size, preferably, the body is in the shape of an elongated cylinder. The body will be about 0.8 to about 5 mm in length, preferably about 1.2 to about 2.5 mm in length. The width of the body will be about 0.2 to about 3, preferably 0.3 to about 1.5 mm. The size of the opening will be from about 1 nm to about 2.5 mm and preferably about 0.15 mm to about 0.8 mm. Instead of one large opening at any one location, multiple small openings may be used. The body of the plug may be wholly or partially transparent or opaque. Optionally, the body may include a tint or pigment that makes the plug easier to see when it is placed in a punctum.

In addition to those already recited here, the body of the devices of the present invention may be made of any suitable biocompatible material including silicone, silicone blends, silicone co-polymers, for example, hydrophilic monomers of polyhydroxyethylmethacrylate (“pHEMA”), polyethylene glycol, polyvinylpyrrolidone, and glycerol, and silicone hydrogel polymers such as, for example, those described in U.S. Pat. Nos. 5,962,548, 6,020,445, 6,099,852, 6,367,929, and 6,822,016, incorporated herein in their entireties by reference. Other suitable biocompatible materials include, for example: polyurethane; polymethylmethacrylate; poly(ethylene glycol); poly(ethylene oxide); polypropylene glycol); poly(vinyl alcohol); poly(hydroxyethyl methacrylate); poly(vinylpyrrolidone) (“PVP”); polyacrylic acid; poly(ethyloxazoline); poly(dimethyl acrylamide); phospholipids, for example, phosphoryl choline derivatives; polysulfobetains; acrylic esters, polysaccharides and carbohydrates, for example, hyaluronic acid, dextran, hydroxyethyl cellulose, hydroxyl propyl cellulose, gellan gum, guar gum, heparan sulfate, chondroitin sulfate, heparin, and alginate; proteins, for example, gelatin, collagen, albumin, and ovalbumin; polyamino acids; fluorinated polymers, for example, PTFE, PVDF, and teflon; polypropylene; polyethylene; nylon; and EVA.

The surface of the devices of the present invention may be wholly or partially coated. The coating may provide one or more of lubriciousness to aid insertion, muco-adhesiveness to improve tissue compatibility, and texture to aid in anchoring the device. Examples of suitable coatings include gelatin, collagen, hydroxyethyl methacrylate, PVP, PEG, heparin, chondroitin sulphate, hyaluronic acid, synthetic and natural proteins, and polysaccharides, thiomers, thiolated derivatives of polyacrylic acid and chitosan, polyacrylic acid, carboxymethyl cellulose and the like and combinations thereof.

Certain exemplary embodiments of the devices of the present invention have a body made of a flexible material that conforms to the shape of whatever it contacts. Optionally, in the punctal plug embodiment, there may be a collarette formed of either a less flexible material than that of the body or material that too conforms to the shape of whatever it contacts. When a punctal plug having both a flexible body and a less flexible collarette is inserted into the lacrimal canaliculus, the collarette rests on the exterior of the lacrimal punctum and the body of the punctal plug conforms to the shape of the lacrimal canaliculus. The reservoir and the body of such punctal plugs are preferably coterminous. That is, the reservoir of such punctal plugs preferably make up the entirety of the body, except for the collarette.

In exemplary embodiments in which one or both of a flexible body and collarette are used, the flexible body and flexible collarette may be made of materials that include nylon, polyethylene terephthalate (“PET”), polybutylene terephthalate (“PBT”), polyethylene, polyurethane, silicone, PTFE, PVDF, and polyolefins. Punctal plugs made of nylon, PET, PBT, polyethylene, PVDF, or polyolefins are typically manufactured, for example, by extrusion forming, injection molding, or thermoforming. Punctal plugs made of latex, polyurethane, silicone, or PTFE are typically manufactured using solution-casting processes.

Processes for manufacturing the punctal plugs useful in the present invention are well known. Typically, the devices are manufactured by injection molding, cast molding, transfer molding, stamping, embossing, or the like. Preferably, the reservoir is filled with one or both of at least one active agent and the active agent-containing material subsequent to the manufacture of the device. Additionally, one or more excipients may be combined with the active agent alone or in combination with the polymeric material.

The amount of active agent used in the devices of the present invention will depend upon the active agent or agents selected, the desired doses to be delivered via the device, the desired release rate, and the melting points of the active agent and active agent-containing material. Preferably, the amount used is a therapeutically effective amount meaning an amount effective to achieve the desired treatment, inhibitory, or prevention effect. Typically, amounts of about 0.05 to about 8,000 micrograms of active agents may be used.

In certain aspects of the present invention, the reservoir may be refilled with a material after substantially all of the active agent-containing material has dissolved or degraded and the active agent is released. For example, the new active agent-containing material may be the same as, or different from, the previous polymeric material, and may comprise at least one active agent that is the same as, or different from the previous active agent. Certain punctal plugs used for particular applications may preferably be refilled with a material while the punctal plugs remain inserted in the lacrimal canaliculus, while other punctal plugs are typically removed from the lacrimal canaliculus, a new material is added, and the punctal plugs are then reinserted into the lacrimal canaliculus.

After the device is filled with the active agent, the plug is sterilized by any convenient method including ethylene oxide, autoclaving, irradiation, and the like and combination thereof. Preferably, sterilization is carried out through gamma radiation or use of ethylene oxide.

The devices described herein may be used to deliver various active agents for the one or more of the treatment, inhibition, and prevention of numerous diseases and disorders. Each device may be used to deliver at least one active agent and may be used to deliver different types of active agents. For example, the devices may be used to deliver azelastine HCl, emadastine difumerate, epinastine HCl, ketotifen fumerate, levocabastine HCl, olopatadine HCl, pheniramine maleate, and antazoline phosphate for one or more of the treatment, inhibition, and prevention of allergies. The devices may be used to deliver mast cell stabilizers, for example, cromolyn sodium, lodoxamide tromethamine, nedocromil sodium, and permirolast potassium.

The devices may be used to deliver mydriatics and cycloplegics including atropine sulfate, homatropine, scopolamine HBr, cyclopentolate HCl, tropicamide, and phenylephrine HCl. The devices may be used to deliver ophthalmic dyes including rose begal, sissamine green, indocyanine green, fluorexon, and fluorescein.

The devices may be used to deliver corticosteroids including dexamethasone sodium phosphate, dexamethasone, fluoromethalone, fluoromethalone acetate, loteprednol etabonate, prednisolone acetate, prednisolone sodium phosphate, medrysone, rimexolone, and fluocinolone acetonide. The devices may be used to deliver non-steroidal anti-inflammatory agents including flurbiprofen sodium, suprofen, diclofenac sodium, ketorolac tromethamine, cyclosporine, rapamycin methotrexate, azathioprine, and bromocriptine.

The devices may be used to deliver anti-infective agents including tobramycin, moxifloxacin, ofloxacin, gatifloxacin, ciprofloxacin, gentamicin, sulfisoxazolone diolamine, sodium sulfacetamide, vancomycin, polymyxin B, amikacin, norfloxacin, levofloxacin, sulfisoxazole diolamine, sodium sulfacetamide tetracycline, doxycycline, dicloxacillin, cephalexin, amoxicillin/clavulante, ceftriaxone, cefixime, erythromycin, ofloxacin, azithromycin, gentamycin, sulfadiazine, and pyrimethamine.

The devices may be used to deliver agents for the one or more of the treatment, inhibition, and prevention of glaucoma including, epinephrines, including, for example, dipivefrin; alpha-2 adrenergic receptors, including, for example, aproclonidine and brimonidine; betablockers including, betaxolol, carteolol, levobunolol, metipranolol, and timolol; direct miotics, including, for example, carbachol and pilocarpine; cholinesterase inhibitors, including physostigmine and echothiophate; carbonic anhydrase inhibitors, including, for example, acetazolamide, brinzolamide, dorzolamide, and methazolamide; prostoglandins and prostamides including latanoprost, bimatoprost, uravoprost, and unoprostone cidofovir.

The devices may be used to deliver antiviral agents, including fomivirsen sodium, foscarnet sodium, ganciclovir sodium, valganciclovir HCl, trifluridine, acyclovir, and famciclovir. The devices may be used to deliver local anesthetics, including tetracaine HCl, proparacaine HCl, proparacaine HCl and fluorescein sodium, benoxinate and fluorescein sodium, and benoxnate and fluorexon disodium. The devices may be used to deliver antifungal agents, including, for example, fluconazole, flucytosine, amphotericin B, itraconazole, and ketocaonazole.

The devices may be used to deliver analgesics including acetaminophen and codeine, acetaminophen and hydrocodone, acetaminophen, ketorolac, ibuprofen, and tramadol. The devices may be used to deliver vasoconstrictors including ephedrine hydrochloride, naphazoline hydrochloride, phenylephrine hydrochloride, tetrahydrozoline hydrochloride, and oxymetazoline. The devices may also be used to deliver vitamins, antioxidants, and nutraceuticals including vitamins A, D, and E, lutein, taurine, glutathione, zeaxanthin, fatty acids and the like.

The active agents delivered by the devices may be formulated to comprise excipients including synthetic and natural polymers, including, for example, polyvinylalcohol, polyethyleneglycol, PAA (polyacrylic acid), hydroxymethyl cellulose, glycerine, hypromelos, polyvinylpyrrolidone, carbopol, propyleneglycol, hydroxypropyl guar, glucam-20, hydroxypropyl cellulose, sorbitol, dextrose, polysorbate, mannitol, dextran, modified polysaccharides and gums, phosolipids, and sulphobetains.

Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims.

What is claimed is:

1. A lacrimal insert for the controlled release of a therapeutic agent into the eye, the lacrimal insert comprising;
a housing having a first end, a second end, and defining cavity, the first end comprising at least one of a rounded or angled cross-sectional profile, and the second end comprising at least one of one or more ribs, notches, protrusions, or generally annular features of greater cross-sectional profile than the remaining portions of the housing;
a drug core positioned within the cavity, the drug core comprising at least one therapeutic agent; and
one or more retention features operatively associated with at least one of the drug core and the cavity.

2. The lacrimal insert of claim 1 wherein the at least one retention feature comprises at least one locking ring disposed between the drug core and an inner surface wall of the cavity.

3. The lacrimal insert of claim 1 wherein the at least one retention feature comprises one or more spiral features disposed between the drug core and an inner surface wall of the cavity.

4. The lacrimal insert of claim 1 wherein an inner surface wall defining the cavity is tapered from the first end toward the second end.

5. The lacrimal insert of claim 4 wherein the inner surface wall defining the cavity includes more than one inward and more than one outward taper.

6. The lacrimal insert according to claim 1, wherein at least one of the drug core and the housing having an anchor head configuration.
